(19) United States
(12) Patent Application Publication
(10) Pub. No.: US 2011/0046541 A1
(43) Pub. Date: Feb. 24, 2011

(54) Phacoemulsification Needle Tip with Interior Step

(76) Inventor: Takayuki Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/019,569

(22) Filed: Jan. 24, 2008

Related U.S. Application Data
(60) Provisional application No. 60/886,792, filed on Jan. 26, 2007.

(51) Int. Cl. A61F 9/007

(52) U.S. Cl. 604/22

(57) ABSTRACT

A phacoemulsification needle is provided for use with a high-frequency phacoemulsification handpiece with the needle tip having a land formed on the interior of the tip proximate the tip opening, creating a “stepped” configuration.

### FIELD OF THE INVENTION

This disclosure relates to surgical instruments and surgical techniques used in eye surgery and more particularly, to phacoemulsification needle tip designs for use with phacoemulsification handpieces.

### PRIORITY

This application claims priority from U.S. Patent Application Ser. No. 60/886,792, filed Jan. 26, 2007, which is incorporated herein by reference.

### BACKGROUND OF THE INVENTION

A common ophthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. For example, it is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

Published U.S. patent application 2006/0217672 (Chon) teaches and describes a phacoemulsification tip having a crimped or swaged tip end.

U.S. Pat. No. 6,077,285 (Boukhny) teaches and describes a torsional ultrasound handpiece configured to impart both longitudinal and torsional motion to a phacoemulsification needle.

U.S. Pat. No. 6,402,769 (Boukhny) is a continuation in part of the '285 patent and further particularizes the frequencies at which the crystals providing both the torsional and longitudinal motion are activated.

I have determined that improved results can be achieved using high-speed handpieces if the phacoemulsification tip is provided with a particular geometry. I have also determined that these improved results can be achieved using the straight phacoemulsification needle configuration, a configuration which is favored by a considerable number of doctors.

In accordance with these criteria, I have designed tips that are specifically configured to enhance the emulsifying effect created by the handpiece.

In accordance with an example of the invention, a phacoemulsification needle is provided for use with a high-frequency phacoemulsification handpiece with the needle tip having a land formed on the interior of the tip proximate the tip opening, creating a “stepped” configuration.

In another example, the tip opening is formed at different angles.

### BRIEF DESCRIPTION OF THE DRAWINGS

These and further aspects of the present invention will be best understood by reference to the accompanying drawings wherein:

FIG. 1 is a drawing showing prior art oval and square-shaped tips;

FIG. 2 is a drawing showing several prior art needle cross-sectional configurations;

FIG. 3 is an end view of a tip constructed in accordance with a preferred embodiment of the present invention;

FIG. 4 is a partial sectional view of the tip shown in FIG. 3 with a beveled configuration; and

FIG. 5 is a partial sectional view of the tip shown in FIG. 3 with a straight tip configuration.

### DETAILED DESCRIPTION OF THE INVENTION

Referring now to FIG. 1, the numeral 10 indicates generally a prior art phacoemulsification needle tip as shown in U.S. Pat. No. 6,007,555. Needle 10 terminates in a mouth 12 defined by a lip 14 at the end of needle body 16, with lip 14 and needle body 16 formed as having an oval cross-section configuration.

Referring to FIG. 1, the numeral 18 indicates generally a prior art phacoemulsification needle tip from U.S. Pat. No. 6,007,555, having a mouth 20 defined by a lip 22 at the end of needle 24. The cross-sectional configuration of needle 18 and mouth 20 is a rectangle.

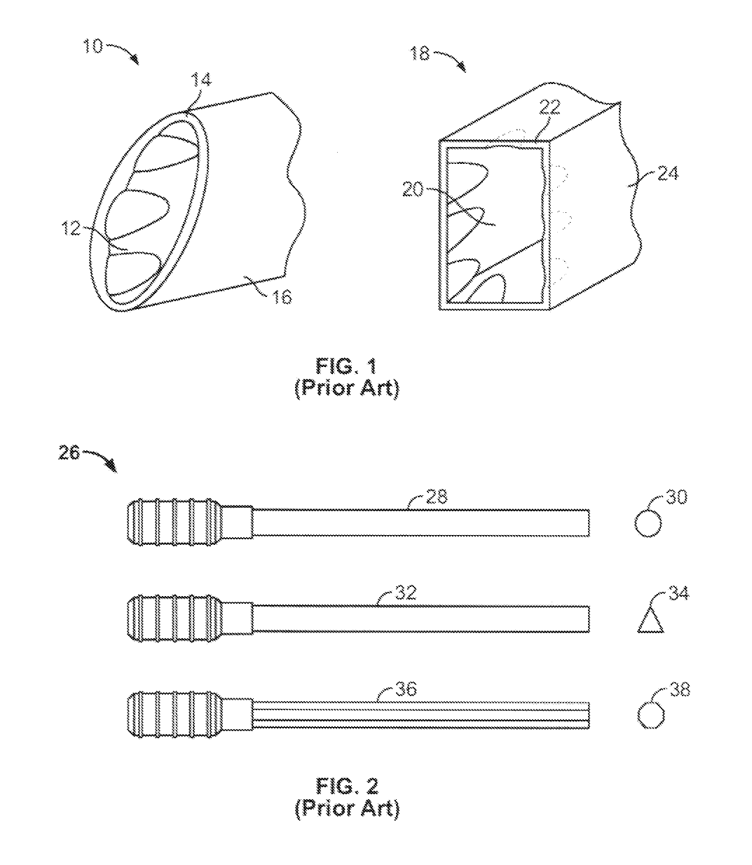

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38.

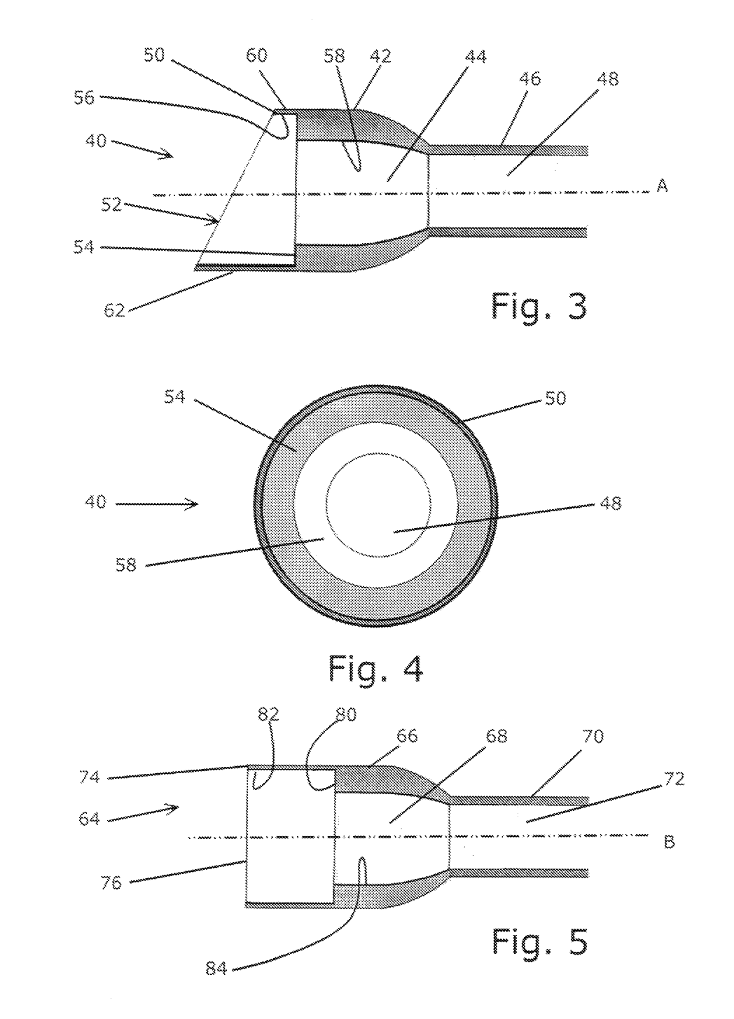

Referring now to FIG. 3, the numeral 40 indicates generally a phacoemulsification needle tip embodying certain aspects of the present invention. In this example, tip 40 is circular in cross-section as seen in FIG. 4 and has a circular wall 42 defining therewithin a cavity 44. Tip 40 is integrally formed with a needle body 46 having a hollow passageway 48 communicating with cavity 44. Wall 42 and cavity 44 define a lip 50 which, in turn, defines opening 52 communicating with cavity 44. In the example shown in FIG. 3, lip 50 is angled with respect to axis A of tip 40 at an angle of about 30°.

As seen in FIGS. 3 and 4, preferably, wall 42 has a first portion 56 having a first thickness and a second portion 58 having a second thickness. In the embodiment shown, the thickness of first wall portion 56 is less than the thickness of second wall portion 58, the result of which is to form a land 54 extending circumferentially within cavity 44 and proximate lip 52. In the configuration shown in FIG. 3, land 54 extends from first inner wall portion 56 to second inner wall portion 58. The configuration in FIG. 3 shows land 54 formed generally perpendicular with axis A although it should be understood that land 54 may be formed at other angles as well. FIG. 3 also illustrates that when lip 52 is formed at a non-perpendicular angle to axis A, first inner wall portion 56 varies in length from a minimum length at 60 to a maximum length at 62, meaning that land 54 is offset by a varying dimension from lip 50.

Referring now to FIG. 5, the numeral 64 identifies generally a phacoemulsification needle tip having an outer wall 66 defining a cavity 68 with tip 64 communicating with a needle body 70 having a central hollow passage 72 communicating with cavity 68.

Wall 66 defines a circular lip 74 which, in turn, defines an opening 76 communicating with cavity 68. As described hereinabove, a first portion 82 of wall 66 extends distally from lip 76 and has a first thickness, and a second portion 84 of wall 66 has a second thickness with said first thickness being less than said second thickness. First and second wall portions 82, 84 meet to form a land 80 positioned within cavity 68 proximate lip 74.

As seen in FIG. 5, lip 74 is perpendicular to axis B of tip 64. The angle at which lip 74 meets axis B may be selected to create a desired efficacy of operation.

As seen in FIG. 5, second wall portion 84 tapers to meet needle body 70. As seen in FIG. 3, second wall portion 58 tapers to meet needle body 46.

Each land 60, 80 defines thereby a cutting or emulsifying surface when tips 40, 64 are used. This is believed to enhance the cutting or emulsifying efficacy of tips 40, 64.

While the foregoing describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

I claim:

1. A phacoemulsification needle for emulsifying body tissue, said needle adapted to be attached to a phacoemulsification hand piece, said needle comprising:
a hollow needle shaft having a distal end and a proximal end,
said needle shaft having a longitudinally-extending central axis;
means formed at said proximal end to mount said needle to said handpiece;
a hollow needle tip formed at said distal end and communicating with said hollow needle shaft,
said tip having a side wall terminating at a lip,
said side wall and said lip defining a needle mouth,
said side wall having at least a first portion with at least a first thickness and a second portion with at least a second thickness,
said first side wall portion extending from said lip,
said first thickness being less than said second thickness,
said first and second side wall portions contiguous one to the other;
said emulsifying means comprising a land formed by said first and second side wall portions.

2. The apparatus as recited in claim 1 wherein said lip is formed at right angles to said axis.

3. The apparatus as recited in claim 1 wherein said lip is formed at a non-perpendicular angle to said axis.

4. The apparatus as recited in claim 1 wherein said land is at a fixed distance from said lip.

5. The apparatus as recited in claim 1 wherein said land is at a variable distance from said lip.

6. The apparatus as recited in claim 1 wherein said land lies in a plane perpendicular to said axis.

7. The apparatus as recited in claim 1 wherein said land lies in a plane non-perpendicular to said axis.

8. A phacoemulsification needle for emulsifying body tissue, said needle adapted to be attached to a phacoemulsification hand piece, said needle comprising:
a hollow needle shaft having a distal end and a proximal end,
said needle shaft having a longitudinally-extending central axis;
means formed at said proximal end to mount said needle to said handpiece;
a hollow needle tip formed at said distal end and communicating with said hollow needle shaft,
said tip having a side wall terminating at a lip,
said side wall and said lip defining a needle mouth,
said side wall having a first portion with a first thickness and a second portion with a second thickness,
said first side wall portion extending from said lip to and coextensive with said second side wall portion,
said first thickness being less than said second thickness;
means formed on the interior of said tip for emulsifying said tissue,
said emulsifying means comprising a land formed by said first and second side wall portions.

9. The apparatus as recited in claim 8 wherein said lip is formed at right angles to said axis.

10. The apparatus as recited in claim 8 wherein said lip is formed at a non-perpendicular angle to said axis.

11. The apparatus as recited in claim 8 wherein said land is at a fixed distance from said lip.

12. The apparatus as recited in claim 8 wherein said land is at a variable distance from said lip.

13. The apparatus as recited in claim 8 wherein said land lies in a plane perpendicular to said axis.

14. The apparatus as recited in claim 8 wherein said land lies in a plane non-perpendicular to said axis.
